(19) United States
(12) Patent Application Publication
(10) Pub. No.: US 2011/0144581 A1
(43) Pub. Date: Jun. 16, 2011

(54) METHOD FOR BONDING LOW-PROFILE RAPID EXCHANGE CATHETER

(76) Inventors: Nathaniel A. Irwin, Bloomington, IN (US); Frank J. Fischer, JR., Bloomington, IN (US)

(21) Appl. No.: 12/635,397

(22) Filed: Dec. 10, 2009

(51) Int. Cl. A61M 25/10; B29C 65/02

(52) U.S. Cl. 604/103; 156/86

(57) ABSTRACT

A low-profile rapid-exchange catheter device having desirable strength between joined proximal and distal portion may be constructed using a method that includes a textile sleeve and a thermoplastic polymer overlay.

### TECHNICAL FIELD

The present application relates to medical catheters, and more specifically to a method for constructing rapid-exchange medical catheters useful in endovascular and other body lumens.

### BACKGROUND

Medical delivery catheters are well known in the art of minimally invasive surgery for introduction of fluids and devices to sites inside a patient's body. For example, balloon dilation of luminal stenoses (e.g., in procedures such as angioplasty or balloon dilation of a bile duct), stent placement, and introduction of radio-opaque contrast fluids are common uses of catheters.

The most widely used form of angioplasty makes use of a dilation catheter having an inflatable balloon at its distal end. In coronary procedures, a hollow guide catheter or wire guide typically is used for guiding the dilation catheter through the vascular system to a position near the stenosis (e.g., to a coronary arterial lumen occluded by plaque). Using fluoroscopy, the physician guides the dilation catheter the remaining distance through the vascular system until a balloon is positioned to cross the stenosis. The balloon is then inflated by supplying pressurized fluid, through an inflation lumen in the catheter, to the balloon. Inflation of the balloon causes a widening of the lumen of the artery to reestablish acceptable blood flow through the artery. In some cases, a stent may be deployed with or instead of the balloon to widen and hold open the occluded arterial lumen.

Preferably a catheter used in endovascular lumens will have several physical characteristics. The profile and shaft size of the dilation catheter should be such that the catheter can reach and cross a very tight stenosis. Portions of the dilation catheter must also be sufficiently flexible to pass through a tight curvature or tortuous passageway, especially in a catheter adapted for use in the coronary arteries. The ability of a catheter to bend and advance effectively through the endovascular or other lumens is commonly referred to as the “trackability of the catheter.” Another important feature of a dilation catheter is its “pushability.” Pushability involves the transmission of longitudinal forces along the catheter from its proximal end to its distal end so that a physician can push the catheter through the vascular or other lumenal system and the stenoses. Effective catheters should be both trackable and pushable.

Two commonly used types of dilation catheters are referred to as “long-wire” catheters and “short-wire” catheters. A long-wire catheter is one in which a wire guide lumen is provided through the length of the catheter that is adapted for use with a wire guide that can first be used to establish the path to and through a stenosis to be dilated. The dilation catheter can then be advanced over the wire guide until the balloon on the catheter is positioned within the stenosis.

In short-wire catheters, the wire guide lumen may not extend the entire length of the catheter. In this type of catheter, the wire guide lumen may extend only from the distal end of the balloon to a point intermediate the distal and proximal ends of the catheter. This shorter lumen is the only portion of the catheter contacting the wire guide. It is sometimes desirable to exchange this first catheter and/or balloon for a second catheter (e.g., to “exchange out” a balloon catheter, and then “exchange in” a stent-deployment catheter). The exchange is preferably executed by leaving the wire guide in place during removal of the first catheter and using it as a guide for the second catheter. The first catheter is withdrawn or otherwise removed over the wire guide, and then a second catheter is introduced over the wire guide.

Short-wire catheters are often easier to exchange than catheters having the wire guide lumen extending the entire length of the catheter. This is because the wire guide need not be as long as a “long wire” configuration, which requires that a length of the wire guide extending outside the patient's body be longer than the portion of the catheter extending over the long wire guide in order for a doctor or assistant to maintain a grasp on the wire guide (to avoid undesired movement or displacement thereof). The short wire guide configuration catheters (known also as “rapid exchange catheters) also create less friction during mounting and exchange operations due to the shorter wire guide lumen, leading to a reduced likelihood of displacing the wire guide.

Catheters for use in endovascular lumens typically require a variation in physical properties along different portions thereof. For example, a certain degree of stiffness is required for pushability and trackability near the proximal end while distal end requires a great deal of flexibility. A catheter having uniform properties throughout its length poses disadvantages in that it is likely to be too proximally flexible or too distally stiff. As a result, most catheter shafts (especially endovascular catheters) are made from multiple materials along the shaft length. For example, a catheter shaft may have a stiffer proximal portion made of metal hypotube and a distal portion made of a more flexible plastic. This combination of materials poses problems of cost and efficiency in construction, and the junctions provide problematic possibilities for structural failure (such as binding, kinking, or even separation) as well as requiring specialized connection means. In a typical catheter embodiment a proximal catheter portion may be a single-lumen construction of about 2.5 Fr, being joined to a distal dual-lumen catheter portion that is about 4 Fr. This construction presents problems for effective, strong bonding of the proximal and distal catheter sections due to the small amount of surface area and limited amount of material present at the joint of the sections. As procedures using catheters of these dimensions may subject them to forces and torque on the order of 15-30 N or greater, catheter devices may fail at this joint.

It would therefore be advantageous to provide a catheter and construction method for making the catheter that includes a strong joint that does not significantly increase the profile (e.g., outer diameter) of the catheter while providing desirable strength at the junction of proximal and distal catheter portions.

### BRIEF SUMMARY

In one aspect the present invention includes a catheter device, adaptable for use in endovascular lumens or other body lumens, that has a construction of a first tubing material for a substantial portion of its proximal length and that is configured for use in a short-wire including that it has a distal dual lumen portion constructed of a second tubing material that may be the same as or different from the first. The method and device embodiments described and claimed herein preferably provide a catheter device having good pushability and trackability while minimizing the likelihood of failure at a joint between the first and second tubing materials. Embodiments of the present invention may be adaptable for a variety of applications (e.g., placement of expandable stents, balloon dilation of stenoses) and use in a variety of surgical locations (e.g., vascular, gastroenterological).

The embodiments herein may be adaptable for use in a variety of minimally invasive surgical treatments (including, e.g., angioplasty, bile duct dilation).

### BRIEF DESCRIPTION OF THE DRAWINGS

FIG. 1 is a partial view of a catheter device, showing an unjoined abutment between a proximal catheter section and a distal catheter section;

FIG. 2 shows the catheter of FIG. 1 with a textile first sleeve or wrap disposed around the joint;

FIG. 3 shows a longitudinal section view of the catheter of FIG. 1 with a second sleeve or wrap disposed around the textile first sleeve or wrap and a heat-shrink tube encompassing that assembly; and

FIG. 4 shows a longitudinal section view of a finished joint of the catheter of FIG. 1.

### DETAILED DESCRIPTION

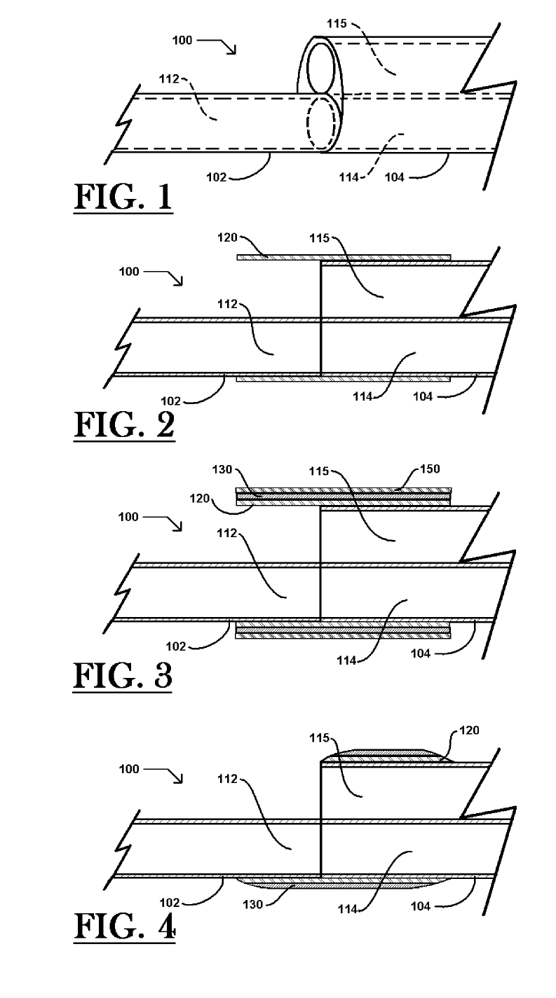

A method for bonding portions of a low-profile rapid-exchange catheter 100 is described with reference to FIGS. 1-4B, and a catheter 100 formed by the method is described with reference thereto. FIG. 1 is a partial view of an unassembled catheter device 100, showing an unjoined abutment between a proximal single-lumen catheter section 102 and a distal dual-lumen catheter section 104. Each of the catheter sections 102, 104 is generally elongate and preferably is sufficiently flexible to be directed through a tortuous passage of a patient body. In most embodiments, the distal section 104 will be shorter in length than the proximal section 102. Exemplary catheter construction materials may include one or more of nylon, polytetrafluoroethylene (PTFE), silicone, polyurethane (PU), polyethylene (PE), and polyvinylchloride (PVC).

The lumen 112 of the proximal section 102 preferably aligns with at least one lumen 114 of the distal section 114 such that it will provide a path for fluid and/or mechanical communication (e.g., passage of a wire guide or other device, passage of inflation fluid to a balloon lumen, or other catheter lumen applications) through the lumens 112, 114. The distal section 104 includes a second lumen 115 configured as a rapid-exchange wire guide lumen that may be configured for passage of a wire guide, including that the wall surrounding the proximal opening of the second lumen 115 may be (as shown) generally transverse relative to the catheter longitudinal axis, or it may be angled relative thereto. The open patency of the lumens 112, 114, 115 may be maintained during the bonding method steps by use of a mandrel (not shown) disposed through one or more of the lumens.

As a first step for bonding the proximal and distal catheter sections 102, 104, a textile material 120 is provided and placed around a circumference of the joint as shown in the longitudinal section view of FIG. 2. The textile 120 may be provided as a strip, sheet, or tube. In one exemplary embodiment joining a 2.5 Fr proximal section 102 with a 4 Fr distal section 104, the textile 120 may be provided as a seamlessly woven polyester tube about 0.05 mm in thickness. This woven construction will provide both longitudinal and torsional strength. Exemplary woven materials may include one or more of PET, Nylon 6-6 or other nylon, polyimide, PE, and (PTFE).

Next, as shown in FIG. 3, a second layer including a thermoplastic material 130 may be wrapped around the textile 120. In the exemplary embodiment, the thermoplastic material 130 may be provided as a tube, sheet, strip, or strand of nylon overlying the textile 120 at a thickness of about 0.18 mm. Then, to facilitate the bonding, a heat-shrink tube 150 may be provided around the textile 120 and thermoplastic material 130 across the joint (the heat-shrink tube may be constructed of one or more of fluorinated ethylene propylene (FEP), other fluoropolymers, polyolefin, polyvinylidene fluoride (PVDF), and chlorinated elastomer (e.g., Neoprene), or another suitable material known to those in the art). In the exemplary embodiment, the layered assembly around the joint may be heated at about 400° C. for about 10 seconds to bond the textile, thermoplastic material, and catheter portions with each other. Thereafter, the heat-shrink tube 150 may be removed leaving the textile/nylon interface 120, 130 intact. Any excess material from the jacketing layers 120, 130 that might obscure the upper lumen 115 may be trimmed away. As will be appreciated, temperature and time for heating the heat shrink sleeve will be determined by the specific material and dimensions of the sleeve, thermoplastic material, and textile, values for which will be understood by those having skill in the art.

It will be appreciated that this method provides a rapid-exchange catheter 100 including desirable strength across the joint between its two portions 102, 104. It should also be appreciated that—even though the layers are shown in FIG. 4 as being relatively thick and well-defined—the final joint will preferably provide at most a slight increase in the outer diameter of the finished joined catheter device 100 including a smooth transition between the joint ends and the outer circumferences of the proximal and distal catheter portions 102, 104. In the exemplary embodiment, the woven polyester tubing will provide desirable tensile strength sufficient to prevent separation of the joint during procedures using the catheter 100 that exert 15-30 N or more of force upon the catheter.

Those of skill in the art will appreciate that other embodiments and variants of the structures and methods described above may be practiced within the scope of the present invention, and that the drawings of different embodiments are not necessarily shown to scale or proportion. It is therefore intended that the foregoing detailed description be regarded as illustrative rather than limiting, and that it be understood that it is the following claims, including all equivalents, that are intended to define the spirit and scope of this invention.

1. A method for bonding a low-profile rapid exchange catheter, the method comprising steps of:
providing a proximal catheter portion including a first lumen and a distal catheter portion comprising a second lumen and a third lumen;
aligning the first and second catheter portions such that an end of each abuts an end of the other to form a joint in a manner providing a fluid-patent path of communication between the first and second lumens, and leaving the third lumen substantially unobstructed by the proximal catheter portion to allow transitory passage of a wire guide therethrough;
placing a woven textile material around a circumference of the joint;
placing a thermoplastic polymer material around the textile material;
disposing a first mandrel through the first lumen and the second lumen;
disposing a second mandrel through the third lumen;
placing a heat-shrink sleeve around the thermoplastic polymer material; and heating the heat shrink sleeve.

2. The method of claim 1, further comprising a step of removing the heat-shrink sleeve.

3. (canceled)

4. The method of claim 1, wherein the textile material comprises a woven polyester tubing.

5. The method of claim 1, wherein the thermoplastic material comprises nylon.

6. A catheter constructed according to the method of claim 1.

7. The catheter of claim 6, wherein the textile material comprises a woven polyester tubing.

8. The catheter of claim 6, wherein the thermoplastic material comprises nylon.

9. The method of claim 1, wherein the step of heating comprises heating at about 400° C. for a predetermined time.

10. A method for bonding a low-profile rapid exchange catheter, the method comprising steps of:
providing a proximal catheter portion including a proximal catheter lumen and a distal catheter portion comprising a first distal catheter lumen and a second distal catheter lumen;
aligning the first and second catheter portions such that an end of each abuts an end of the other to form a joint in a manner providing a fluid-patent path of communication between the proximal catheter lumen and one of the distal catheter lumens, and leaving an other of the distal catheter lumens substantially unobstructed by the proximal catheter portion to allow transitory passage of a wire guide therethrough;
placing a seamless tube of woven polyester around a circumference of the joint;
placing nylon around the polyester tube;
disposing a first mandrel through the proximal catheter lumen and the one of the distal catheter lumens;
disposing a second mandrel through the other of the distal catheter lumens;
placing a heat-shrink sleeve around the thermoplastic polymer material; and heating the heat shrink sleeve at a predetermined temperature.

11. The method of claim 10, further comprising a step of removing the heat-shrink sleeve.

12. A catheter device constructed according to the method of claim 10.
